(19) United States
(12) Patent Application Publication
(10) Pub. No.: US 2024/0366363 A1
(43) Pub. Date: Nov. 7, 2024

(54) VENOUS STENT

(71) Applicant: Shin-Kong Wu Ho-Su Memorial Hospital, Taipei City (TW)

(72) Inventor: Chia-Hsun LIN, Taipei City (TW)

(21) Appl. No.: 18/355,655

(22) Filed: Jul. 20, 2023

(30) Foreign Application Priority Data
May 2, 2023 (TW) 112116346

(51) Int. Cl. A61F 2/07

(52) U.S. Cl. CPC A61F 2/07

(57) ABSTRACT

A venous stent comprising a first vascular stent and a second vascular stent is provided, in which the first and the second vascular stents with the appropriate diameters can be selected depending on patient's veins and overlappingly combination one to another. Therefore, the venous stent of the present disclosure is preferably suitable for vascular stenosis or obstruction at or close to the confluence of veins, and can improve the matching of the stent with different patients and increase the postoperative problems caused by the inappropriate placement of stent.

### CROSS REFERENCE TO RELATED APPLICATIONS

The non-provisional patent application claims priority to Taiwan patent application with serial number TW 112116346 filed on May 2, 2023. This and all other extrinsic materials discussed herein are incorporated by reference in their entirety.

### BACKGROUND

### Technology Field

The present disclosure is related to a venous stent, especially a stent graft designed to enhance vascular patency at the branching or confluence points of veins.

### Description of Related Art

For a long time, a patient with chronic renal failure have been required to undergo kidney dialysis treatment. In kidney dialysis or other treatments, the use of a central venous catheter for intubation is often necessary to facilitate the kidney dialysis treatment or other treatment. However, based on clinical experience, approximately 25% to 40% of chronic renal dialysis patients suffer from central venous stenosis or obstruction. Since the venous obstruction occurs near the heart, severe swelling and discomfort gradually develop in the entire hand, forearm, and upper arm. In some cases, the skin may become ulcerated, and the wound healing becomes difficult. Until now, intravascular balloon angioplasty has been the main therapy for central venous obstruction. However, the recurrent stenosis is a common postoperative sequela. At this stage, the vascular wall loses its elasticity, becomes fibrotic, and tends to develop intimal hyperplasia.

In current surgical treatment, a single stent is commonly used to treat venous obstruction. However, this approach has several drawbacks when dealing with obstructions located near venous confluences. For example, as shown in FIG. 1A, if the venous obstruction A is located at the distal end of the right subclavian vein (61), near its confluence with the right internal jugular vein (63), the traditional treatment using a single stent involves bridging between the right subclavian vein (61) and the right brachiocephalic vein (62) to ensure smooth blood flow reflux in the right subclavian vein (61), alleviate symptoms like swelling and cyanosis in the right arm, and prevent necrosis and amputation of the right arm (as seen in FIG. 1B). However, this bridging method sacrifices the smooth blood flow of the right internal jugular vein (63). In addition, consider FIG. 1A as an example. If the venous obstruction B is located at the distal end of the left subclavian vein (65), where it joins the left internal jugular vein (66), another traditional method for treating venous obstruction with a single stent involves placing the stent at the site of venous obstruction B in the left subclavian vein (65). The proximal end of the stent is slightly extended at the confluence (as shown in FIG. 1B) to avoid the stent's opening causing the retraction of the outer vascular wall at the site of stent expansion during stent placement (i.e., the inner retraction of the vascular opening at the confluence), which could affect the blood flow in the left subclavian vein (65). However, since the stent extends into the confluence, this bridging method also affects the smooth blood flow of the left internal jugular vein (66). Similarly, when treating compression stenosis at the junction of the inferior vena cava and the iliac veins, the traditional method of single stent placement also encounters similar problems.

In addition, since the size of the proximal and distal vessels at the site of venous obstruction varies among patients, the treatment of venous obstruction requires the selection of a stent that matches the size of the affected vessels. However, cardiovascular stent manufacturers tend to produce standardized stents to avoid increasing manufacturing costs and inventory pressure. Consequently, due to individual differences and variations in the location of venous obstruction, some patients may struggle to find standardized stents that match the diameters of both ends of the affected blood vessels. If incomplete standardized stents that do not fully match the diameters of both ends of the affected blood vessels, are used, postoperative issues can arise, such as stent detachment or displacement, due to improper stent sizing. Therefore, finding suitable stents that match the diameters of various patient blood vessels is an important issue that needs to be addressed.

The present disclosure provides a venous stent comprising a first vascular stent and a second vascular stent, which can be selected based on the vascular structure of the affected area in each patient to match the appropriate diameters of the first and second stents and overlap and assemble them. Therefore, the stent combination of the present disclosure can improve the compatibility of the stent with different patients at various locations, maintain the patency of the tributary vessels at the confluence, reduce postoperative complications, and alleviate inventory pressure on manufacturers, ultimately leading to cost reduction.

### SUMMARY

The present disclosure provides a venous stent expandable from a constrained configuration to a depolyed configuration comprising a first vascular stent composed of a tubular graft member and a plurality of supports surrounding the tubular graft member; a fitting member fixed on the inner wall of tubular graft member of the first vascular stent; and a second vascular stent composed of a tubular graft member and a plurality of support surrounding the tubular graft member, and the second vascular stent comprises a anchored end and a fitted end connected into the fitting member.

In one aspect, the present disclosure provides a method for placing the venous stent of the present disclosure, and the method comprisies
providing a venous stent deployment device and loading with the venous stent of the present disclosre;
delivering the first vascular stent wrapped in the first sheath to a target location in a first vein with the venous stent deployment device, and retracting the first sheath to expand the first vascular stent from the constrained configuration to the deployed configuration for placement at the target location in the first vein; and
delivering the second vascular stent wrapped in the second sheath to a target location in a second vein with the venous stent deployment device and connecting the fitted end into the fitting member of the first vascular stent, and retracting the second sheath to expand the second vascular stent from the constrained configuration to the deployed configuration for disposing the fitted end of the second vascular stent in the fitting member of the first vascular stent and fixing the anchored end at the target location in the second vein.

In another aspect, the present disclosure provides a method for trearing obstruction at a venous confluence, and the method comprisies
providing a venous stent deployment device loading with the first vascular stent of the present disclosure in a constrained configuration;
delivering the first vascular stent in a constrained configuration to a target location in a first vein with the venous stent deployment device, and retracting the first sheath to expand the first vascular stent from the constrained configuration to the deployed configuration for placement at the target location in the first vein;
loading with the second vascular stent of the present disclosure in a constrained configuration on the venous stent deployment device; and
delivering the second vascular stent in a constrained configuration to a target location in a second vein with the venous stent deployment device and connecting the fitted end into the fitting member of the first vascular stent, and retracting the second sheath to expand the second vascular stent from the constrained configuration to the deployed configuration for disposing the fitted end of the second vascular stent in the fitting member of the first vascular stent and fixing the anchored end at the target location in the second vein.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIGS. 1A and 1B are schematic diagrams of an exemplary venous obstruction and vascular stent placement in the prior art, respectively.

FIG. 2 is a schematic diagram of a venous stent in a depolyed configuration according to the present disclosure.

FIG. 3 is a schematic diagram of the venous stents of the present disclosure in a constrained configuration.

FIGS. 4A-4D show the different exemplary configurations of the fitting member of the venous stent of the present disclosure.

FIG. 5 is a schematic diagram of a configuration of the venous stent of the present disclosure.

FIG. 6 is a schematic diagram of the fitting member of the venous stent of the present disclosure in the form of a balloon-dilatation stent configuration.

FIG. 7 is a schematic diagram of a configuration of the venous stent disclosed in the present disclosure.

FIG. 8 is a schematic diagram of the venous stent of the present disclosure being placed at the confluence of the right subclavian vein and the right brachiocephalic vein.

FIG. 9 is a schematic diagram of the venous stent of the present disclosure being placed at the confluence of the left common iliac vein and the inferior vena cava.

### DETAILED DESCRIPTION OF THE DISCLOSURE

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains.

### Definitions

As used herein, the articles “a” and “an” refer to one or to more than one (i.e., to at least one) of the grammatical object of the article. By way of example, “a fixed member” means one fixed member or more than one fixed member.

As used herein, the term “constrained configuration” refers to a configuration in which the vascular stent of the present disclosure is compressed and constrained by the sheath or membrane against the central axis (i.e., the mandrel) of the stent into a configuration having a smaller diameter, and such vascular stent can be surgically delivered intravenously to a target site, such as a venous obstruction, using a stent deployment device. As used herein, the term “depolyed configuration” means that the vascular stent of the present disclosure in the constrained configuration can be firmly placed at the target site of the vein due to self-expansion or balloon dilatation after removing components such as the sheath or the membrane.

As used herein, the term “stent graft” refers to a vascular stent composed of a metal stent covered with a special tubular membrane material (such as, polytetrafluoroethylene, polyester, polyurethane, etc.), that is, it consists of metal stents wraped with artificial vascular graft materials.

As used herein, the term “self-expansion” stent means that the vascular stent will expand to the desired diameter when it is not constrained by a sheath or membran. In other words, the stent can be released by pulling back or removing the constraining sheath or membran after delivery and placement at the vascular lesion.

As used herein, the term “balloon-dilatation” stent means that the vascular stent does not expand before it is expanded with a balloon, and thus the stent can be expanded to a desired diameter by balloon dilatation after it is delivered and placed at the vascular lesion.

As used herein, the term “central vein” refers to the large veins surrounding the heart, including internal jugular vein, subclavian vein, brachiocephalic vein, superior vena cava, inferior vena cava, iliac vein, etc.

As used herein, the term “human body acceptable” means that it usually does not cause an allergic reaction, rejection, inflammatory response or the like after the application to a human body.

As used herein, the terms “upstream” and “downstream” of blood vessel are determined by the direction of blood stream. In the case of veins, since the venous blood stream from the peripheral vein to the heart, at one relative position of the blood vessel, such as at the vascular obstruction, the side of peripheral vein is referred to “upstream”, and the side of the heart is referred to “downstream”. In the case of arteries, since the arterial blood stream from the heart to the peripheral artery, at one relative position of the blood vessel, such as at the vascular obstruction, the side of the heart is referred to “upstream”, and the side of peripheral artery is referred to “downstream”.

As used herein, the “proximal end” described for the venous stent placed in the blood vessel refers to the end of the venous stent close to the heart after the venous stent is placed at the target site in the vein. The “distal end” described for the venous stent refers to the end of the venous stent away from the heart or the another end opposite to the “proximal end” after the venous stent is placed at the target site within the vein.

With respect to reference orientation of the various operation steps described herein, the term “proximal” and “distal” are relative to the perspective of the surgeon, who is manipulating the delivery system of the disclosure to deploy the implants described herein. Accordingly, those features of the delivery system held by the surgeon's hand are at the “proximal” side and the assembled system and the implant, initially in its compressed configuration, is located at the “distal” side of the delivery system.

It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 8 to 14 should be considered to have specifically disclosed subranges such as from 8 to 9, from 8 to 10, from 8 to 11, from 9 to 10, from 9 to 11, from 10 to 14, etc., as well as individual numbers within that range, for example, 8, 9, 10, 10.5, 11, 12.6, 13.2 and 14. This applies regardless of the breadth of the range.

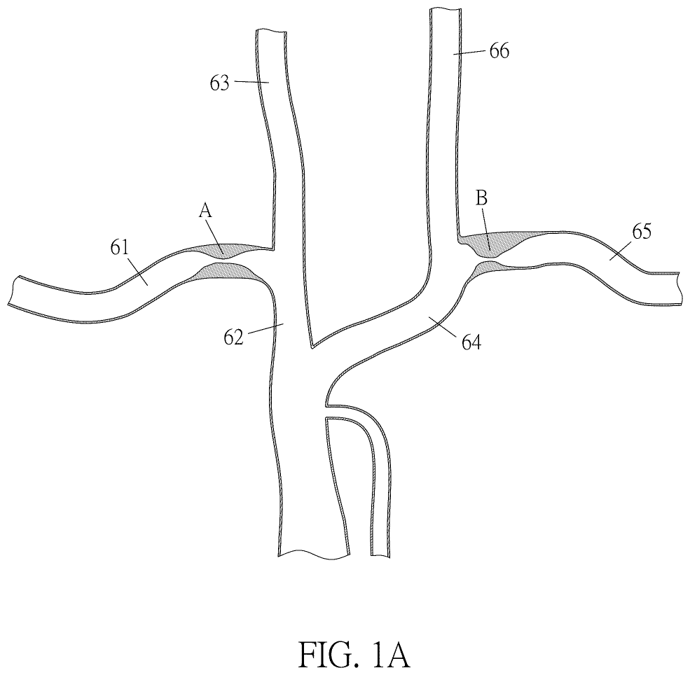

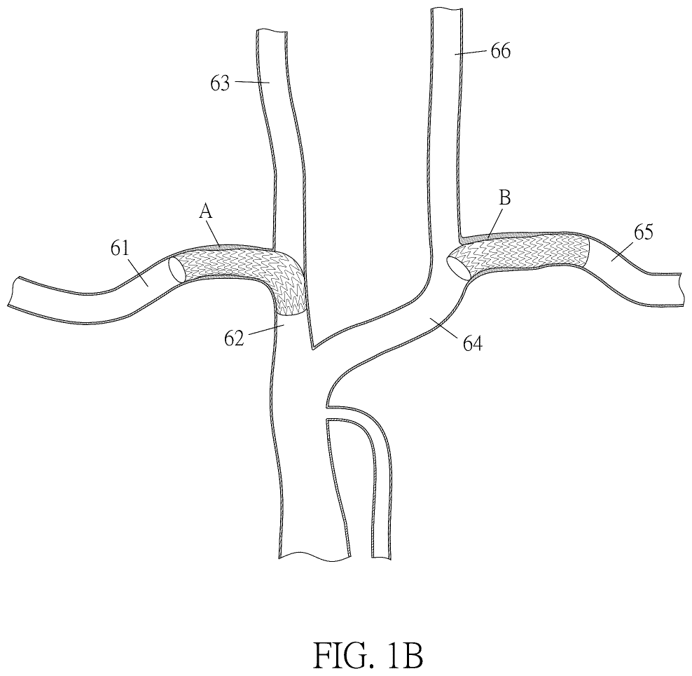

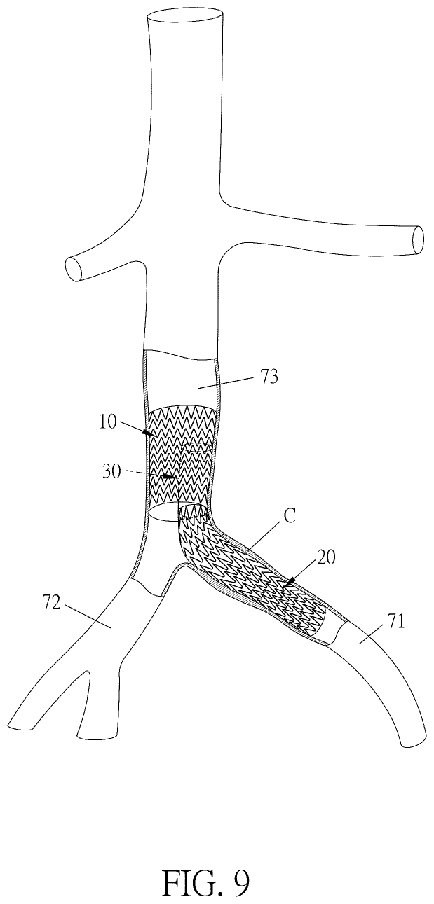

The examples of the venous stent grafts according to the present disclosure is illustrated in detail with reference to FIG. 1˜FIG. 9. These examples are provided for the purpose of illustration only, and the disclosure is not limited to these examples.

### Venous Stent

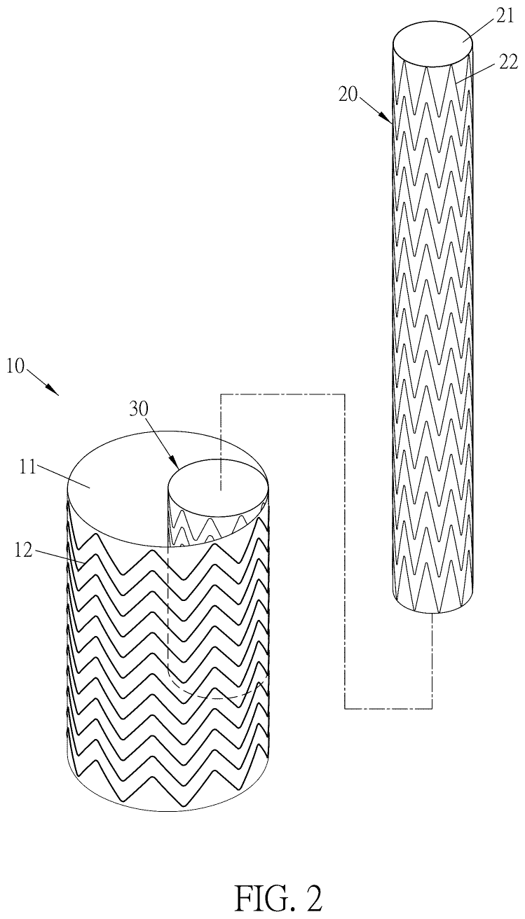

Referring to FIG. 2, it is a schematic diagram of a venous stent in a depolyed configuration according to the present disclosure. The exemplary venous stent comprises a first vascular stent 10 and a second vascular stent 20, wherein the first vascular stent 10 comprises a fitting member 30 connected to the inner wall of the first vascular stent 10. The first vascular stent 10 and the second vascular stent 20 are stent grafts. Both the first vascular stent 10 and the second vascular stent 20 comprise a tubular graft member (11, 21) and a plurality of supports (12, 22). The fitting member 30 is fixed on the inner wall of the tubular graft member 11 of the first vascular stent 10.

In one embodiment, the first stent 10 and the second stent 20 are stent grafts composed of a graft member wrapped around a plurality of supports. The graft member may be a flexible tubing manufactured by a human body acceptable polymeric medical material, and the polymeric medical material is a blood-impervious flexible film or fabric manufactured by materials such as, but not limited to, polytetrafluoroethylene (PTFE), polyamide, polyester fiber, polypropylene and the like. Preferably, the materials like polytetrafluoroethylene or a polyester such as polyethylene terephthalate (PET) are used.

In one embodiment, the supports may be fabricated from the same or different materials acceptable for the human body. For example, the supports can be fabricated from superelastic materials or plastically-deformable materials. In another embodiment, the supports are fabricated from superelastic materials, such as Nitinol (nickel-titanium elastic alloy), medical stainless steel alloy, or an alloy comprising iron, cobalt, nickel, chromium, molybdenum and the like. In another embodiment, the supports are fabricated from plastically-deformable materials, such as 316L stainless steel. Since the flow rate of blood in the venous vessel is slower than that in the arterial vessel, the stent in the venous vessel needs to have improved ability to resist thrombosis. Therefore, the supports of venous stent in the present disclosure can preferredly have one or more surface coatings. For example, the supports can be spraied, sputterred or deposited with a surface coating material, such as, titanium dioxide, titanium nitride, titanium carbide, biomolecular groups, and so on. Additionally, the configuration of the supports surrounding the tubular graft member may be spiral, wavy, jagged, repeating zigzag, curved, or any other shape. They can also collectively form a reticular structure.

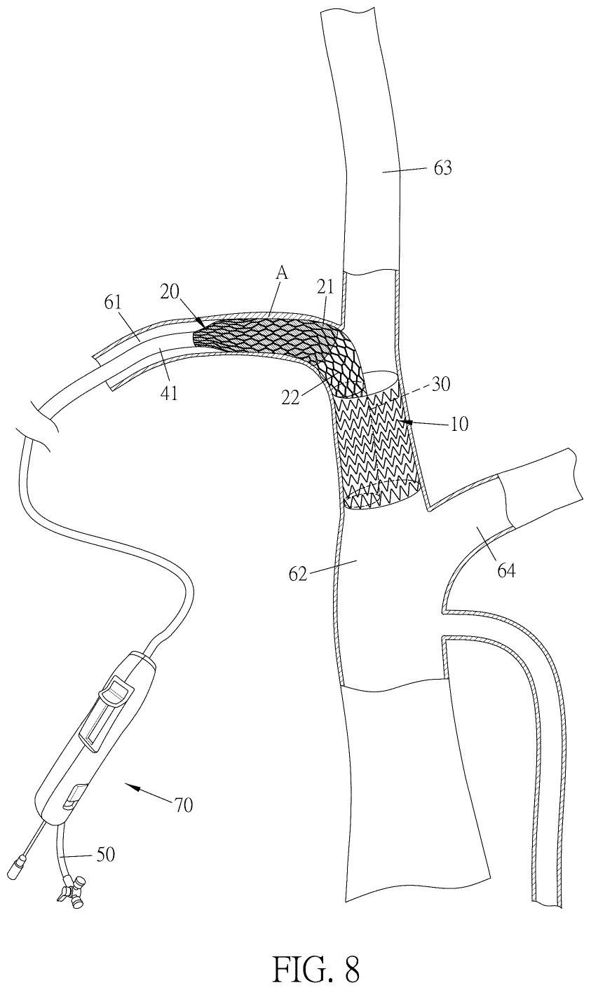

In one embodiment, a plurality of supports of the first vascular stent and the second vascular stent can form a mesh structure. In one preferred embodiment, a plurality of supports of the second vascular stent can form a mesh structure, and the length of the supports in the mesh structure may be longer than that of the tubular graft member. In one preferred embodiment, the fitted end of the second vascular stent does not cantain the tubular graft member. Such a configuration helps reduce obstruction to blood flow at the venous confluence (as shown in FIG. 8).

In the venous stent of the present disclosure, the diameters of both ends of the first vescular stent are not particularly limited and can be manufactured in accordance with the size of vein in the patient's affected region, as long as the first vascular stent can be firmly placed at the target site in the blood vessel. In one embodiment, the diameters of both ends of the first vascular stent may range from about 12 to about 36 mm, respectively. In another embodiment, when the venous stent of the present disclosure is applied near the superior vena cava, the diameters of both ends of the first vascular stent may preferably range from about 12 to about 24 mm, respectively. For example, each end can be 12, 14, 16, 18, 20, 22 or 24 mm, and the upper limit of the diameter can be determined in accordance with the individual patient. In another embodiment, when the venous stent of the present disclosure is applied near the inferior vena cava, the diameters of both ends of the first vascular stent may preferably range from about 16 to about 36 mm, respectively. For example, each end can be 16, 18, 20, 22, 24, 26, 28, 30, 32, 34 or 36 mm, and the upper limit of the diameter can be determined in accordance with the individual patient. Additionally, the length of the first vascular stent ranges from about 20 to about 70 mm, such as 20, 25, 30, 35, 40, 45, 50, 55, 60, 65 or 70 mm.

In the venous stent of the present disclosure, the diameters of both ends of the second vescular stent are not particularly limited and can be manufactured in accordance with the size of vein in the patient's affected region, as long as the blood flowing through the vascular stenosis or obstruction can be guided into the second vascular stent without infiltrating into the gap between the second vascular stent and the vessel wall. In one embodiment, the diameters of both ends of the second vascular stent may range from about 8 to about 22 mm, respectively. In another embodiment, when the venous stent of the present disclosure is applied near the superior vena cava, the diameters of both ends of the second vascular stent may preferably range from about 8 to about 16 mm, respectively. For example, each end can be 8, 10, 12, 14 or 16 mm, and the upper limit of the diameter can be determined in accordance with the individual patient. In another embodiment, when the venous stent of the present disclosure is applied near the inferior vena cava, the diameters of both ends of the second vascular stent may preferably range from about 10 to about 22 mm, respectively. For example, each end can be 10, 12, 14, 16, 18, 20, or 22 mm, and the upper limit of the diameter can be determined in accordance with the individual patient. Additionally, the length of the second vascular stent ranges from about 40 to about 160 mm, such as 40, 60, 80, 100, 120, 140, or 160 mm.

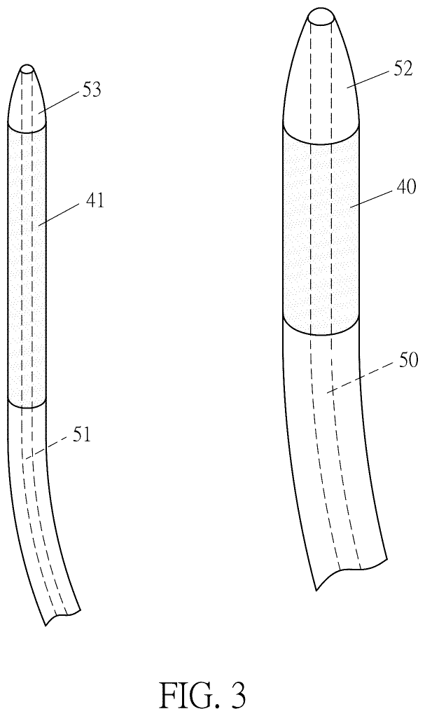

Referring to FIG. 3, it is a schematic diagram of the venous stents of the present disclosure in a constrained configuration. A first sheath 40 is configurated to wrap and constrain a first vascular stent and the fitting member in constrained configurations against the mandrel. A second sheath 41 is configurated to wrap and constrain a second vascular stent in a constrained configuration against the mandrel. In one embodiment, under this constrained configuration, the first stent and the second stent are delivered and placed between the inner catheter (50, 51) and the sheath (40, 41), respectively. In such a compressed arrangement, the supports are positioned close to each other.

The first sheath 40 and the second sheath 41 can be formed from suitable polymeric materials, such as nylon (polyamide), urethane, polypropylene, as well as polyamide co-polymers (such as polyether block amides (PEBAX®)), or other materials that may be employed.

In a preferred embodiment of the present disclosure, the first vascular stent 10 and the second vascular stent 20 are self-expansion stent-grafts. When the first sheath 40 is retracted proximally, the first vascular stent 10 can gradually be revealed. The part of the first vascular stent 10 unconstrained by the first sheath 40 will expand itself into a depolyment configuration (as shown in FIG. 2). Similarly, when the second sheath 41 is retracted proximally, the second vascular stent 20 can be revealed gradually. The part of the second vascular stent 20 unconstrained by the first sheath 41 will expand itself into a depolyed configuration (as shown in FIG. 2).

In one embodiment, the front ends of the inner catheters (50, 51) can respectively have a top guide head (52, 53), that are used to guide the venous stent in the vein and position it at the target site during the venous stent placement. The top guide heads (52, 53) can be a solid structure or have a hollow structure connectable with the inner catheters (50, 51), and can be used to introduce other surgical equipment during the venous stent placement process.

### Fitting Member

Referring to FIG. 2, the first vascular stent 10 of the venous stent of the present disclosure includes a fitting member 30, which is fixed on the inner wall of the tubular graft member 11 of the first vascular stent 10. When combining the venous stent of the present disclosure, the fitting member 30 is used to fit the proximal end of the second vascular stent 20, that is, the fitting end. The term “fitting” here means that when the second vascular stent 20 is inserted into the fitting member 30, its proximal end will not slip out of the first vascular stent 10, nor will it move inside the first vascular stent 10 to disturb the blood flow, and thus the fitting between the second vascular stent 20 and the fitting member 30 is not necessary to achieve a completely tight seal without blood leakage. Therefore, the diameter or shape of the fitting member 30 does not need to closely match the diameter of the fitting end of the second vascular stent 20. In other words, one size of the fitting member 30 can be used for a variety of second vessel stents 20 having different diameters or sizes. Additionally, the length of the fitting member 30 may be equal to or less than the length of the first vascular stent 10 For example, it can be equal to the length of the first vascular stent 10 to half of the length of the first vascular stent 10.

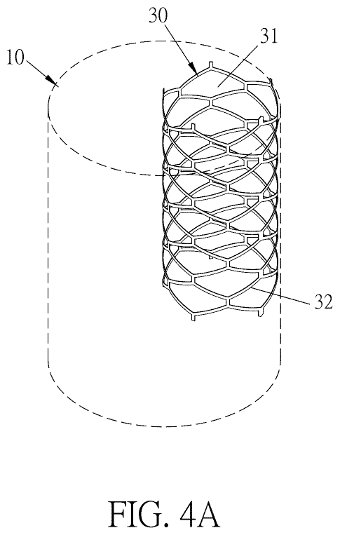

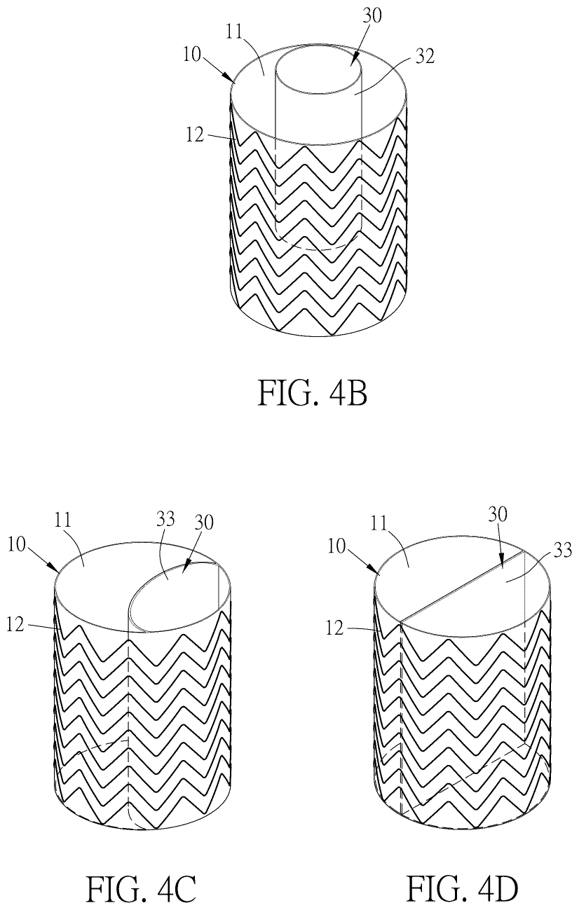

The configuration of the fitting member 30 can be identical with that of the first vascular stent 10 and/or the second vascular stent 20, such as a tubular graft member wrapped by a plurality of supports (as shown in FIG. 2). In one embodiment, the fitting member 30 may be a stent graft. Additionally, the configuration of the fitting member 30 may be different from that of the first vascular stent 10 and the second vascular stent 20. For instance, FIGS. 4A-4D show different exemplary configurations of the fitting member 30.

The fitting member 30 shown in FIG. 4A is a tubular metal stent composed solely of mesh-like supports. The mesh-like supports can be a self-expansion fitting member made of superelastic materials or a balloon-dilatation fitting member made of plastically-deformable materials. In one embodiment, the fitting member 30 is a self-expansion fitting member, which is composed of a mesh-like supports made of superelastic materials, such as nickel-titanium alloy (Nitinol) and the like. In the case that the first vascular stent is a self-expanding stent, when the sheath wrapping the first vascular stent is removed, the first vascular stent and the fitting member 30 will automatically expand to the deployed configuration simultaneously. In another embodiment, the fitting member 30 is a balloon-dilatation fitting member, which is composed of a mesh-like supports made of plastically-deformable materials, such as 316L stainless steel and the like. In the scenario where the first vascular stent is a self-expanding stent, when the sheath wrapping the first vascular stent is removed, the first vascular stent will automatically expand to the deployed configuration. The operator or surgeon can then utilize balloon inflation to expand the fitting member 30 to the desired diameter. In other words, the fitting member 30 made of plastically-deformable materials can be expanded with a balloon to achieve an appropriate diameter that corresponds to the diameter of the matched second vascular stent within the limit of plastic deformation. In the preferred embodiment, the fitting member 30 is a mesh-like supports made of plastically-deformable materials.

FIGS. 4B to 4D show a fitting member 30 composed of a tubular or sheet artificial vascular membrane. The artificial vascular membrane can be made from the same material for manufacturing the tubular graft member in the first vascular stent 10 and the second vascular stent 20. For instance, the fitting member 30 can be a flexible tubes made of polymeric medical materials acceptable for use in the human body, such as, but not limited to, polytetrafluoroethylene (PTFE), polyamide, polyester fiber, polypropylene and the like. Preferably, materials such as polytetrafluoroethylene or a polyester like polyethylene terephthalate (PET) are used.

FIG. 4B shows a fitting member 30 composed of tubular artificial vascular membrane. The outer wall of the fitting member 30 is fixed to the inner wall of the tubular graft member 11 of the first vascular stent 10.

FIGS. 4C and 4D show a fitting member 30 composed of a sheet artificial vascular membrane 33 and a part of the tubular graft member 11 of the first vascular stent 10. Both sides of the sheet artificial vascular membrane 33 are respectively fixed at two places on the inner wall of the tubular graft member 11 of the first vascular stent 10, so that the interior of the first vascular stent 10 can be partitioned into the inner passage area of the first vascular stent 10 and the fitting member 30. The size of the fitting member 30 can be selected according to the diameter of the second vascular stent to be dispolyed. For example, if a smaller-diameter second vascular stent is used, the first vascular stent 10 having the configuration of the fitting member 30 as shown in FIG. 4C can be used. If a larger-diameter second vascular stent is used, the first vascular stent 10 having the configuration of the fitting member 30 as shown in FIG. 4D can be used.

Referring to the fitting member 30 as shown in FIGS. 4B to 4D, in the case where the first vascular stent 10 is a self-expanding stent, once the sheath wrapping the first vascular stent 10 is removed, the first vascular stent 10 will automatically expand to the deployed configuration. Subsequently, the second vascular stent wrapped by the sheath in a constrained configuration is delivered into the fitting member 30 using a surgical instrument. An appropriate length of the fitted end of the second vascular stent is then placed within the fitting member 30, enabling the placement of the second vascular stent (FIGS. 4B to 4D are not shown, please refer to FIG. 8).

### Processes for the Combination and Placement of the Venous Stent

The present disclosure provides a method for placing the aforementioned venous stent, and the method comprisies:
providing a venous stent deployment device that can be loaded with the aforementioned first vascular stent or the second vascular stent of the venous stent, wherein the first vascular stent and the second vascular stent are in the form of constrained configuration, and the venous stent deployment device can be a vascular stent deployment device known in the art or commonly used in surgery;
delivering the first vascular stent wrapped in the first sheath to a target location in a first vein with the venous stent deployment device, and retracting the first sheath to expand the first vascular stent from the constrained configuration to the deployed configuration for placement at the target location in the first vein; and
delivering the second vascular stent wrapped in the second sheath to a target location in a second vein with the venous stent deployment device and connecting the fitted end into the fitting member of the first vascular stent, and retracting the second sheath to expand the second vascular stent from the constrained configuration to the deployed configuration for disposing the fitted end of the second vascular stent in the fitting member of the first vascular stent and fixing the anchored end at the target location in the second vein.

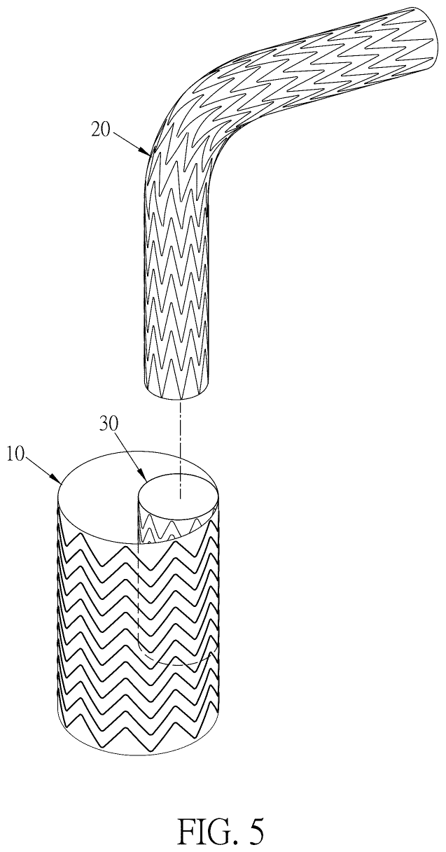

In one embodiment, the second vascular stent of the venous stent of the present disclosure is designed in a curved configuration, which can be adapted to the specific vein tissue structure where it is positioned. Referring to FIG. 5, the utilization of the curved configuration of the second vascular stent in combination with the first vascular stent can enhance blood flow in areas such as venous obstructions A or B illustrated in FIG. 1A.

In one embodiment, the fitting member of the venous stent of the present disclosure is a self-expansion stent member. When the first sheath wrapping the first vascular stent is retracted, both the fitting member and the first vascular stent will expand simultaneously, as they are no longer constrained by the first sheath.

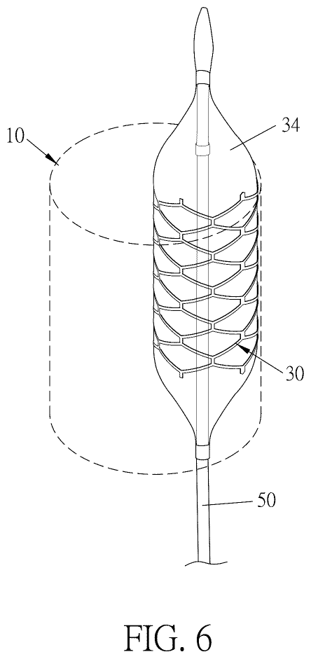

FIG. 6 is a schematic diagram of the fitting member of the venous stent of the present disclosure in the form of a balloon-dilatation stent configuration. The first vascular stent 10 is equipped with a balloon 34 positioned between the fitting member 30 and the inner catheter 50 for expanding the fitting member. Once the first vascular stent 10 is expanded from its constrained configuration to the deployed configuration and placed at the target site of the first vein, the balloon 34 can be inflated by the inner catheter 50 to expand the fitting member 30 to the desired diameter, that is, the appropriate diameter of the fitted end of the second vascular stent can be fitted.

### Application of Venous Stent

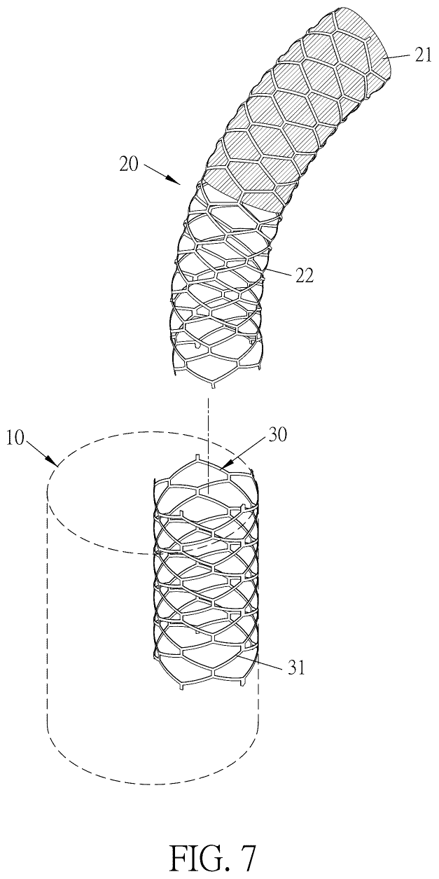

FIG. 7 is a schematic diagram of a configuration of the venous stent disclosed in the present disclosure, wherein the fitting member 30 is made by supports 31 in the form of a reticular structure manufactured by plastically-deformable materials, and the plurality of supports 22 of the second vascular stent 20 also form a reticular structure. The length of the reticular-structure supports 22 can be longer than the tubular graft member 21. The length of the tubular graft member 21 and the length of the reticular-structure supports 22 may be determined according to the distance between the veous obstruction and the first vascular stent positioned at the vein confluence. For example, the anchored end of the second vascular stent 20 includes a tubular graft member 21 (depicted in gray) wrapped by the reticular-structure supports 22, wherein the length of tubular graft member 21 is approximately half to two-thirds of the total length of the second vascular stent 20. In other words, the fitted end of the second vascular stent 20 only includes the reticular-structure supports 22, and its length is approximately half to one-thirds of the total length of the second vascular stent 20.

FIG. 8 is a schematic diagram of the venous stent of the present disclosure being placed at the confluence of the right subclavian vein and the right brachiocephalic vein. Referring to FIGS. 7 and 8, venous obstruction A is located at the end of the right subclavian vein 61, near its confluence with the right brachiocephalic vein 62. When placing the venous stent of the present dislcosure, first, the appropriate diameter of the first vascular stent 10 is selected according to the vascular diameter of the patient's right brachiocephalic vein 62. Then, the first vascular stent 10 is delivered along the right subclavian vein 61 with the stent deployment device 70 to the downstream of the confluence of the right internal jugular vein 63 and the right subclavian vein 61 in the right brachiocephalic vein 62. The first sheath (not shown) wrapping the first vascular stent 10 is retracted, allowing the first vascular stent 10 to self-expand into a deployed configuration and fix itself at a predetermined position within the right brachiocephalic vein 62. The fitting member 30 is expanded to a desired diameter by inflating a balloon (not shown) through the inner catheter 50. The balloon is deflated and retracted. Subsequently, the second vascular stent 20 with appropriate length and diameter is selected and delivered along the right subclavian vein 61 with the stent deployment device 70. The fitted end of the second vascular stent 20 is placed into the fitting member 30 of the first vascular stent 10 disposed in the right brachiocephalic vein 62. The second sheath 41 wrapping the second vascular stent 20 is retracted, and thereby the second vascular stent 20 can self-expand into a deployed configuration, wherein the fitted end remains within the fitting member 30, while the anchored end is fixed to the right subclavian vein 61.

After the placement of the venous stent of the present disclosure, the blood flow at the venous obstructio A can be restored, thereby preventing or improving the swelling of the patient's right upper limb due to venous obstruction. Additionally, the first vascular stent 10 maintains a pathway for blood in the right internal jugular vein 63 to flow back to the heart. Furthermore, the fitted end of the second vascular stent 20 is only composed of supports 22 in the form of a reticular structure, that is, there is no tubular graft member. This design minimizes the impact on blood flow in the internal jugular vein.

FIG. 9 is a schematic diagram of the venous stent of the present disclosure placed at the confluence of the left common iliac vein and the inferior vena cava. Referring to FIG. 9, venous obstruction C is located at the end of the left common iliac vein71, near its confluence with the right common iliac vein72. Firstly, when placing the venous stent, the first vascular stent 10 with an appropriate diameter is selected according to the vascular diameter of the patient's inferior vena cava 73, and the first vascular stent 10 is delivered along the left common iliac vein 71 with the stent deployment device (not shown) to the downstream of the confluence of the left common iliac vein 71 and right common iliac vein (72) in the inferior vena cava 73. The first sheath (not shown) wrapping the first vascular stent 10 is retracted, allowing the first vascular stent 10 and the fitting member 30 to self-expand into deployed configurations and the first vascular stent 10 to fix itself at a predetermined position in the inferior vena cava 73. Subsequently, the second vascular stent 20 with appropriate length and diameter is selected and delivered along the left common iliac vein 71 with the stent deployment device it's the fitted end of the second vascular stent 20 is placed into the fitting member 30 of the first vascular stent 10 disposed in the inferior vena cava 73. The second sheath (not shown) wrapping the second vascular stent 20 is retracted, and thereby the second vascular stent 20 can self-expand into a deployed configuration, wherein the fitted end remains within the fitting member 30, while the anchored end is fixed to the left common iliac vein 71.

After the placement of the venous stent of the present disclosure is disposed, the blood flow at the venous obstructio C can be restored, thereby preventing or improving the swelling of the patient's left lower limb due to venous obstruction. Additionally, the second vascular stent 20 can guide blood of the left common iliac vein 71 into the inferior vena cava 73, and the first vascular stent 10 maintains a pathway for blood in the right common iliac vein 72 to flow back to the heart. This design minimizes the impact of the venous stent on blood flow in the right common iliac vein 72 and the inferior vena cava 73.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure can be extended easily with appropriate modifications to other applications.

### Enumerated Embodiments

The following exemplary embodiments are provided, the numbering of which is not to be construed as designating levels of importance.

Embodiment 1 provides a venous stent expandable from a constrained configuration to a depolyed configuration comprising
a first vascular stent composed of a tubular graft member and a plurality of supports surrounding the tubular graft member,
a fitting member fixed on the inner wall of tubular graft member of the first vascular stent, and
a second vascular stent composed of a tubular graft member and a plurality of support surrounding the tubular graft member, and the second vascular stent comprises a anchored end and a fitted end connected into the fitting member.

Embodiment 2 provides the venous stent of the Embodiment 1, wherein the first vascular stent and the second vascular stent are self-expansion stents.

Embodiment 3 provides the venous stent of the Embodiment 2, which further comprises a first sheath and a second sheath, wherein the first sheath is configurated to compress and constrain the first vascular stent and the fitting member in constrained configurations against the mandrel, and the second sheath is configurated to wrap and constrain the second vascular stent in a constrained configuration against the mandrel.

Embodiment 4 provides the venous stent of the Embodiment 1, wherein the length of the first vascular stent ranges from about 20 to about 70 mm.

Embodiment 5 provides the venous stent of the Embodiment 1, wherein the diameters of the two ports of the first vascular stent range from about 12 to about 36 mm, respectively.

Embodiment 6 provides the venous stent of the Embodiment 1, wherein the length of the second vascular stent ranges from about 40 to about 160 mm.

Embodiment 7 provides the venous stent of the Embodiment 1, wherein the diameters of the two ports of the second vascular stent range from about 8 to about 22 mm, respectively.

Embodiment 8 provides the venous stent of the Embodiment 1, wherein the second vascular stent is in a curved configuration.

Embodiment 9 provides the venous stent of the Embodiment 1, wherein the support is in the shape of a spiral, a wave, a zigzag, a repeating zigzag, a curved line or a mesh structure.

Embodiment 10 provides the venous stent of the Embodiment 1, wherein the plurality of supports of the second vascular stent form a mesh structure, and the fitted end does not contain the tubular graft member.

Embodiment 11 provides the venous stent of the Embodiment 1, wherein the length of the fitting member is equal to or less than the length of the first vascular stent.

Embodiment 12 provides the venous stent of the Embodiment 1, wherein the fitting member is a self-expansion stent member, a balloon-dilatation stent member, or a tubular graft member.

Embodiment 13 provides the venous stent of the Embodiment 3, wherein the fitting member is a balloon-dilatation stent member, and the first vascular stent further comprises a balloon disposed in and used to expand the fitting member, and the balloon is the fitting member, and the first sheath is configurated to compress and constrain the first vascular stent, the fitting member and the balloon in constrained configurations against the mandrel.

Embodiment 14 provides the venous stent of the Embodiment 1, wherein the fitting member is a diaphragm member, and the two sides of the diaphragm member are respectively fixed on the inner wall of the tubular graft member of the first vascular stent.

Embodiment 15 provides the venous stent of the Embodiment 1, wherein the fitting member is a diaphragm member, and two sides of the diaphragm member are fixed on the inner wall of the tubular graft member of the first vascular stent to form a tube shape.

Embodiment 16 provides a method for placing the venous stent of any one of the Embodiments 1 to 15, and the method comprisies
providing a venous stent deployment device that can be loaded with the venous stent of any one of Embodiments 1 to 15;
delivering the first vascular stent wrapped in the first sheath to a target location in a first vein with the venous stent deployment device, and retracting the first sheath to expand the first vascular stent from the constrained configuration to the deployed configuration for placement at the target location in the first vein; and
delivering the second vascular stent wrapped in the second sheath to a target location in a second vein with the venous stent deployment device and connecting the fitted end into the fitting member of the first vascular stent, and retracting the second sheath to expand the second vascular stent from the constrained configuration to the deployed configuration for disposing the fitted end of the second vascular stent in the fitting member of the first vascular stent and fixing the anchored end at the target location in the second vein.

Embodiment 17 provides the method of the Embodiment 16, wherein the fitting member is a self-expansion stent member, and the fitting member expands simultaneously with the first vascular stent when the first sheath is retracted.

Embodiment 18 provides the method of the Embodiment 16, wherein the fitting member is a balloon-dilatation stent member, and the first vascular stent further comprises a balloo disposed in and used to expand the fitting member, wherein the fitting membe is expanded by inflating the balloon after the first vascular stent is expended from constrained configuration to the depolyed configuration for placement at the target location in the first vein.

Embodiment 19 provides a method for trearing obstruction at a venous confluence, and the method comprisies
providing a venous stent deployment device loading with the first vascular stent in a constrained configuration according to the Embodiment 3,
delivering the first vascular stent in a constrained configuration to a target location in a first vein with the venous stent deployment device, and retracting the first sheath to expand the first vascular stent from the constrained configuration to the deployed configuration for placement at the target location in the first vein,
loading with the second vascular stent in a constrained configuration according to the Embodiment 3 on the venous stent deployment device, and
delivering the second vascular stent in a constrained configuration to a target location in a second vein with the venous stent deployment device and connecting the fitted end into the fitting member of the first vascular stent, and retracting the second sheath to expand the second vascular stent from the constrained configuration to the deployed configuration for disposing the fitted end of the second vascular stent in the fitting member of the first vascular stent and fixing the anchored end at the target location in the second vein.

Embodiment 20 provides the method of the Embodiment 19, wherein the second vascular stent is in a curved configuration.

Embodiment 21 provides the method of the Embodiment 19, wherein the fitting member is a balloon-dilatation stent member, and the first vascular stent further comprises a balloon disposed in and used to expand the fitting member, and the balloon is the fitting member, and the first sheath is configurated to compress and constrain the first vascular stent, the fitting member and the balloon in constrained configurations against the mandrel.

Embodiment 22 provides the method of the Embodiment 19, wherein the fitting member is a diaphragm member, and the two sides of the diaphragm member are respectively fixed on the inner wall of the tubular graft member of the first vascular stent.

Embodiment 23 provides the method of the Embodiment 19, wherein the fitting member is a diaphragm member, and both sides of the diaphragm member are fixed on the inner wall of the tubular graft member of the first vascular stent to form a tube shape.

Embodiment 24 provides the method of the Embodiment 19, wherein the obstruction is in the central veins.

Embodiment 25 provides the method of the Embodiment 19, wherein the the first vascular is brachiocephalic vein, and the second vein is subclavian vein.

Embodiment 26 provides the method of the Embodiment 19, wherein the first vascular is inferior vena cava, and the second vein is common iliac vein.

What is claimed is:

1. A venous stent expandable from a constrained configuration to a depolyed configuration comprising
a first vascular stent composed of a tubular graft member and a plurality of supports surrounding the tubular graft member,
a fitting member fixed on the inner wall of tubular graft member of the first vascular stent, and
a second vascular stent composed of a tubular graft member and a plurality of support surrounding the tubular graft member, and the second vascular stent comprises a anchored end and a fitted end connected into the fitting member.

2. The venous stent of claim 1, wherein the first vascular stent and the second vascular stent are self-expansion stent grafts.

3. The venous stent of claim 2 further comprises a first sheath and a second sheath, wherein the first sheath is configurated to compress and constrain the first vascular stent and the fitting member in constrained configurations against the mandrel, and the second sheath is configurated to wrap and constrain the second vascular stent in a constrained configuration against the mandrel.

4. The venous stent of claim 1, wherein the length of the first vascular stent ranges from 20 mm to 70 mm.

5. The venous stent of claim 1, wherein the diameters of the two ports of the first vascular stent range from 12 mm to 36 mm, respectively.

6. The venous stent of claim 1, wherein the length of the second vascular stent ranges from 40 mm to 160 mm.

7. The venous stent of claim 1, wherein the diameters of the two ports of the second vascular stent range from 8 mm to 22 mm, respectively.

8. The venous stent of claim 1, wherein the second vascular stent is in a curved configuration.

9. The venous stent of claim 1, wherein the support is in the shape of a spiral, a wave, a zigzag, a repeating zigzag, a curved line or a mesh structure.

10. The venous stent of claim 1, wherein the plurality of supports of the second vascular stent form a mesh structure, and the supports in the form of the mesh structure are longer than the tubular graft member.

11. The venous stent of claim 10, wherein the fitted end of the second vascular stent is only composed of the supports in the form of the mesh structure.

12. The venous stent of claim 11, wherein the length of the supports at the fitted end is ½ to ⅓ of the total length of the second vascular stent.

13. The venous stent of claim 1, wherein the length of the fitting member is equal to or less than the length of the first vascular stent.

14. The venous stent of claim 1, wherein the fitting member is a self-expansion stent member, a balloon-dilatation stent member, or a tubular graft member.

15. The venous stent of claim 3, wherein the fitting member is a balloon-dilatation stent member, and the first vascular stent further comprises a balloon disposed in and used to expand the fitting member, and the balloon is the fitting member, and the first sheath is configurated to compress and constrain the first vascular stent, the fitting member and the balloon in constrained configurations against the mandrel.

16. The venous stent of claim 1, wherein the fitting member is a diaphragm member, and the two sides of the diaphragm member are respectively fixed on the inner wall of the tubular graft member of the first vascular stent.

17. The venous stent of claim 1, wherein the fitting member is a diaphragm member, and two sides of the diaphragm member are fixed on the inner wall of the tubular graft member of the first vascular stent to form a tube shape.
